(19) United States
(12) Patent Application Publication
(10) Pub. No.: US 2024/0016415 A1
(43) Pub. Date: Jan. 18, 2024

(54) METHOD AND SYSTEM FOR CONDUCTING INTERACTIVE REHABILITATION SESSIONS WITH CONTINUOUS MONITORING

(71) Applicant: PES University, Bengaluru (IN)

(72) Inventors: Shrikar Madhu, Bengaluru (IN); Kruthika Suresh, Banglore (IN); Yousha Mahamuni, Banglore (IN)

(21) Appl. No.: 18/352,300

(22) Filed: Jul. 14, 2023

(30) Foreign Application Priority Data
Jul. 15, 2022 (IN) 202241040652

(51) Int. Cl. A61B 5/11; A61B 5/00; G16H 20/30; G16H 15/00

(52) U.S. Cl. CPC A61B 5/1128; A61B 5/7264; G16H 20/30; A61B 5/7475; A61B 5/1116; A61B 5/112; G16H 15/00

(57) ABSTRACT

The present invention discloses a method and system for conducting interactive rehabilitation session for a user with continuous monitoring. The session for user is generated in form of tasks, using current condition of the user and/or recommendations provided by a physiotherapist. The tasks may be performed by the patient in virtual environment, using, one or more virtual objects. Further, the tasks performed by the user is continuously monitored by implementing image processing techniques. The monitoring is performed to check if the user is performing the tasks as recommended by the physiotherapist. In case a deviation is detected, dynamic feedbacks may be provided to the user in real-time to correct actions of the user. Further, upon ending the sessions, the user may be scored or graded for the session based on performance of the tasks.

### BACKGROUND

### Technical Field

The present disclosure relates to the field of conducting rehabilitation sessions for patients, and in particular, relates to a system and method for conducting interactive rehabilitation sessions with continuous monitoring of the patients.

### Description of the Related Art

Rehabilitation therapies help to restore, maintain, and make the most of a patient's mobility, function, and well-being. Such rehabilitation therapies may be physical rehabilitation like physiotherapy or neurological rehabilitation which aid in recovery from an injury or a disorder and encompasses various treatment modalities such as massages, heat therapy, exercises, electrotherapy, patient education, and advice for treating an injury, disorder, ailment, or deformity. Typically, physical rehabilitation sessions are conducted for the recovery of the patient but the physical rehabilitation sessions have limitations at least in terms of accessibility, convenience, flexibility, cost-effectiveness, continuity of care, privacy, and comfort.

Due to the above-mentioned limitation of the physical rehabilitation session, online sessions are provisioned to patients in which a session (such as a live session or a pre-recorded session) may be communicated to the patient. Such sessions may include physical activities or exercises to be performed with corresponding instructions on how to perform such physical activities or exercises. Some of the conventional systems teach to monitor the patient during the session. Typically, in such a conventional system, a trainer or a physiotherapist may continuously monitor the patient during the session. Further, in some cases, 3D cameras, motion-detecting sensors, and/or other hardware sensors may be used to monitor the patients during the session. However, such systems are unable to yield the same results as the physical session at least due to the non-immersive nature of such sessions, inaccurate monitoring of the patients while performing the exercises during the sessions, and limited interaction between the trainer/physiotherapist and the patient. Such limitations of the online sessions over the physical sessions are heightened when the session is a rehabilitation session for an injured patient because accurate and continuous monitoring is very important in the rehabilitation sessions and the continuous interaction is required to avoid the worsening of the injury.

Thus, there is a need for an improved system and method for conducting interactive rehabilitation sessions with continuous monitoring of the patients to overcome the drawbacks of the conventional systems.

### BRIEF SUMMARY

One or more embodiments are directed to a system and method for conducting interactive rehabilitation sessions with continuous monitoring. The system discloses the generation of rehabilitation sessions for a user (such as a patient or an athlete) in the form of one or more tasks based on current condition of the user and/or recommendations provided by a trainer/physiotherapist of the user. Further, the system facilitates the user to perform one or more tasks in a virtual environment using one or more virtual objects. Such one or more tasks provides an immersive experience to the user to engage with the rehabilitation session actively and with more interest. Also, the one or more tasks performed by the user during the rehabilitation session is continuously monitored using camera of user device (such as mobile phone, laptop, or television) by implementing image processing techniques. The monitoring enables checking if the user is correctly performing the one or more tasks as recommended by the physiotherapist or not. In case a deviation is detected during the monitoring, one or more dynamic feedbacks may be provided to the user in real-time for provisioning interactivity in the rehabilitation sessions and correcting pose, motion, or movement of the user to correctly perform the one or more tasks. Additionally, upon ending the rehabilitation session, the patient may be scored or graded based on performance of the one or more tasks to encourage the user.

An embodiment of the present disclosure discloses the system for conducting interactive rehabilitation sessions with continuous monitoring. The system includes a receiver module to receive, via a user device, one or more inputs from a user in response to one or more questions associated with ailments and requirements pertaining to the user.

Further, the system includes a user engagement module to create and render a customized workout plan to the user for initiating the rehabilitation session. Such customized workout plan is either created automatically or by a physiotherapist based on the received one or more inputs. The customized workout plan includes one or more tasks along with specific attributes and parameters for performing the one or more tasks. The one or more tasks may be associated with sequential instructions to perform physical exercises and/or virtual objects for provisioning physiotherapy. Further, the customized workout plan includes one or more standard clinical features that may be related to required posture and movement from the user for implementing the customized workout plan. In an embodiment, the customized workout plan may be rendered in a virtual environment and may, without any limitation, include one or more virtual objects to facilitate interaction with the user to make the rehabilitation session immersive.

The system also includes a data collection module to receive metrics and movement data of the user while implementing the customized workout plan during the rehabilitation session. In order to receive the metrics and movement data, the data collection module may be communicatively coupled to one or more sensors and/or a camera while implementing the customized workout plan by processing the received one or more inputs. In an embodiment, the metrics and movement data may, without any limitation, include speed, reaction time, angle, distance, stability, jerk, range of motion, flexibility, balance, strength, muscle power, and degree of flexion

Furthermore, the system includes a progress and performance analysis module to extract one or more clinical features based on the received metrics and movement data of the user by coordinating, smoothing, normalization, dimensionality reduction, temporal correlation, windowing, feature extraction, or a combination thereof. The progress and performance analysis module also compares the extracted one or more clinical features with the standard one or more clinical features to determine if there are deviations. In an embodiment, the one or more clinical features are related to actual posture and movement of the user while implementing the customized workout plan. Upon determining the deviations, the progress and performance analysis module may create a report based on the results of the comparison and send the created report to the user and/or a physiotherapist of the user.

Additionally, the system includes a feedback module to provide, by employing an explainable Artificial Intelligence (AI) model, one or more dynamic feedbacks to the user in real-time based on the determined deviations, wherein the one or more dynamic feedbacks are associated with correction of posture and/or movement to overcome the determined deviation.

In an embodiment, the system may additionally include a sandbox to facilitate the physiotherapist to create a new custom exercise with customized constraints to modify the workout plan of the user. The new custom exercise may be created by uploading a video performing the new custom exercise and adding body point of interest, relevant angles, and/or necessary metrics for tracking recovery progress of the user in the rehabilitation session.

An embodiment of the present disclosure discloses the method for conducting interactive rehabilitation sessions with continuous monitoring. The method includes the steps of receiving, via a user device, one or more inputs from a user in response to one or more questions associated with ailments and requirements pertaining to the user. Further, the method includes the steps of creating and render a customized workout plan to the user for initiating the rehabilitation session. The customized workout plan is created based on the received one or more inputs and includes one or more standard clinical features. The method also includes the steps of receiving metrics and movement data of the user while implementing the customized workout plan during the rehabilitation session. Also, the method includes the steps of extracting one or more clinical features based on the received metrics and movement data of the user by coordinating, smoothing, normalization, dimensionality reduction, temporal correlation, windowing, feature extraction, or a combination thereof. Additionally, the method includes the steps of comparing the extracted one or more clinical features with the standard one or more clinical features to determine if there are deviations. Thereafter, the method includes the steps of providing, by employing an explainable Artificial Intelligence (AI) model, one or more dynamic feedbacks to the user in real-time based on the determined deviations. The one or more dynamic feedbacks are associated with correction of posture and/or movement to overcome the determined deviation.

In an embodiment, the method includes the steps of creating a report based on the results of the comparison and sending the report to the user and/or a physiotherapist of the user. Further, the method includes the steps of facilitating the physiotherapist to create a new custom exercise with customized constraints to modify the workout plan of the user. The new custom exercise may be created by uploading a video performing the new custom exercise and adding body point of interest, relevant angles, and/or necessary metrics for tracking recovery progress of the user in the rehabilitation session.

The Features and advantages of the subject matter here will become more apparent in light of the following detailed description of selected embodiments, as illustrated in the accompanying FIGUREs. As will be realized, the subject matter disclosed is capable of modifications in various respects, all without departing from the scope of the subject matter. Accordingly, the drawings and the description are to be regarded as illustrative in nature.

### BRIEF DESCRIPTION OF THE DRAWINGS

In the figures, similar components and/or features may have the same reference label. Further, various components of the same type may be distinguished by following the reference label with a second label that distinguishes among the similar components. If only the first reference label is used in the specification, the description is applicable to any one of the similar components having the same first reference label irrespective of the second reference label.

FIG. 1 illustrates a block diagram of an environment of a system for conducting interactive rehabilitation sessions with continuous monitoring, in accordance with various embodiments of the present disclosure.

FIG. 2 illustrates a block diagram of the system for conducting interactive rehabilitation sessions with continuous monitoring, in accordance with various embodiments of the present disclosure.

FIG. 3 illustrates an overall working mechanism for the system for conducting interactive rehabilitation sessions with continuous monitoring, in accordance with various embodiments of the present disclosure.

FIGS. 4A-4B illustrate an exemplary embodiment of a task for conducting interactive physiotherapy session with continuous monitoring, in accordance with various embodiments of the present disclosure.

FIG. 5 illustrate another exemplary embodiment of a task for conducting interactive physiotherapy session with continuous monitoring, in accordance with various embodiments of the present disclosure.

FIG. 6 illustrates a flowchart of a method for conducting interactive rehabilitation sessions with continuous monitoring, in accordance with an embodiment of the present disclosure.

FIG. 7 illustrates an exemplary computer system in which or with which embodiment of the present disclosure may be utilized.

Other features of embodiments of the present disclosure will be apparent from accompanying drawings and detailed description that follows.

### DETAILED DESCRIPTION

The detailed description set forth below in connection with the appended drawings is intended as a description of exemplary embodiments in which the presently disclosed process can be practiced. The term “exemplary” used throughout this description means “serving as an example, instance, or illustration,” and should not necessarily be construed as preferred or advantageous over other embodiments. The detailed description includes specific details for providing a thorough understanding of the presently disclosed method and system. However, it will be apparent to those skilled in the art that the presently disclosed process may be practiced without these specific details. In some instances, well-known structures and devices are shown in block diagram form to avoid obscuring the concepts of the presently disclosed method and system

Embodiments of the present disclosure include various steps, which will be described below. The steps may be performed by hardware components or may be embodied in machine-executable instructions, which may be used to cause a general-purpose or special-purpose processor programmed with the instructions to perform the steps. Alternatively, steps may be performed by a combination of hardware, software, firmware, and/or by human operators.

Embodiments of the present disclosure may be provided as a computer program product, which may include a machine-readable storage medium tangibly embodying thereon instructions, which may be used to program the computer (or other electronic devices) to perform a process. The machine-readable medium may include, but is not limited to, fixed (hard) drives, magnetic tape, floppy diskettes, optical disks, compact disc read-only memories (CD-ROMs), and magneto-optical disks, semiconductor memories, such as ROMs, PROMs, random access memories (RAMs), programmable read-only memories (PROMs), erasable PROMs (EPROMs), electrically erasable PROMs (EEPROMs), flash memory, magnetic or optical cards, or other types of media/machine-readable medium suitable for storing electronic instructions (e.g., computer programming code, such as software or firmware).

Various methods described herein may be practiced by combining one or more machine-readable storage media containing the code according to the present disclosure with appropriate standard computer hardware to execute the code contained therein. An apparatus for practicing various embodiments of the present disclosure may involve one or more computers (or one or more processors within the single computer) and storage systems containing or having network access to a computer program(s) coded in accordance with various methods described herein, and the method steps of the disclosure could be accomplished by modules, routines, subroutines, or subparts of a computer program product.

### Terminology

Brief definitions of terms used throughout this application are given below.

The terms “connected” or “coupled”, and related terms are used in an operational sense and are not necessarily limited to a direct connection or coupling. Thus, for example, two devices may be coupled directly, or via one or more intermediary media or devices. As another example, devices may be coupled in such a way that information can be passed there between, while not sharing any physical connection with one another. Based on the disclosure provided herein, one of ordinary skill in the art will appreciate a variety of ways in which connection or coupling exists in accordance with the aforementioned definition.

If the specification states a component or feature “may”, “can”, “could”, or “might” be included or have a characteristic, that particular component or feature is not required to be included or have the characteristic.

As used in the description herein and throughout the claims that follow, the meaning of “a,” “an,” and “the” includes plural reference unless the context dictates otherwise. Also, as used in the description herein, the meaning of “in” includes “in” and “on” unless the context dictates otherwise.

The phrases “in an embodiment,” “according to one embodiment,” and the like generally mean the particular feature, structure, or characteristic following the phrase is included in at least one embodiment of the present disclosure and may be included in more than one embodiment of the present disclosure. Importantly, such phrases do not necessarily refer to the same embodiment.

Exemplary embodiments will now be described more fully hereinafter with reference to the accompanying drawings, in which exemplary embodiments are shown. This disclosure may, however, be embodied in many different forms and should not be construed as limited to the embodiments set forth herein. These embodiments are provided so that this disclosure will be thorough and complete and will fully convey the scope of the disclosure to those of ordinary skill in the art. Moreover, all statements herein reciting embodiments of the disclosure, as well as specific examples thereof, are intended to encompass both structural and functional equivalents thereof. Additionally, it is intended that such equivalents include both currently known equivalents as well as equivalents developed in the future (i.e., any elements developed that perform the same function, regardless of structure).

Thus, for example, it will be appreciated by those of ordinary skill in the art that the diagrams, schematics, illustrations, and the like represent conceptual views or processes illustrating systems and methods embodying this disclosure. The functions of the various elements shown in the figures may be provided through the use of dedicated hardware as well as hardware capable of executing associated software. Similarly, any switches shown in the figures are conceptual only. Their function may be carried out through the operation of program logic, through dedicated logic, through the interaction of program control and dedicated logic, or even manually, the particular technique being selectable by the entity implementing this disclosure. Those of ordinary skill in the art further understand that the exemplary hardware, software, processes, methods, and/or operating systems described herein are for illustrative purposes and thus, are not intended to be limited to any particular named.

A system and method for conducting interactive rehabilitation sessions with continuous monitoring is disclosed. The system discloses generation of rehabilitation sessions for a user (such as patient or an athlete) in form of one or more tasks (such as exercise, plucking of flower, and bursting of balloons) based on current condition of the user and/or recommendations provided by a trainer/physiotherapist of the user. Further, the system facilitates the user to perform one or more tasks in a virtual environment using one or more virtual objects (such as the resistance strings, flowers, and balloons). Such one or more tasks provides an immersive experience to the user to engage with the rehabilitation session actively and with more interest. Also, the one or more tasks performed by the user during the rehabilitation session is continuously monitored using camera of user device (such as mobile phone, laptop, or television) by implementing image processing techniques. The monitoring enables checking if the user is correctly performing the one or more tasks as recommended by the physiotherapist or not. In case a deviation is detected during the monitoring, one or more dynamic feedbacks may be provided to the user in real-time for provisioning interactivity in the rehabilitation sessions and correcting pose, motion, or movement of the user to correctly perform the one or more tasks. Additionally, upon ending the rehabilitation session, the patient may be scored or graded based on performance of the one or more tasks to encourage the user.

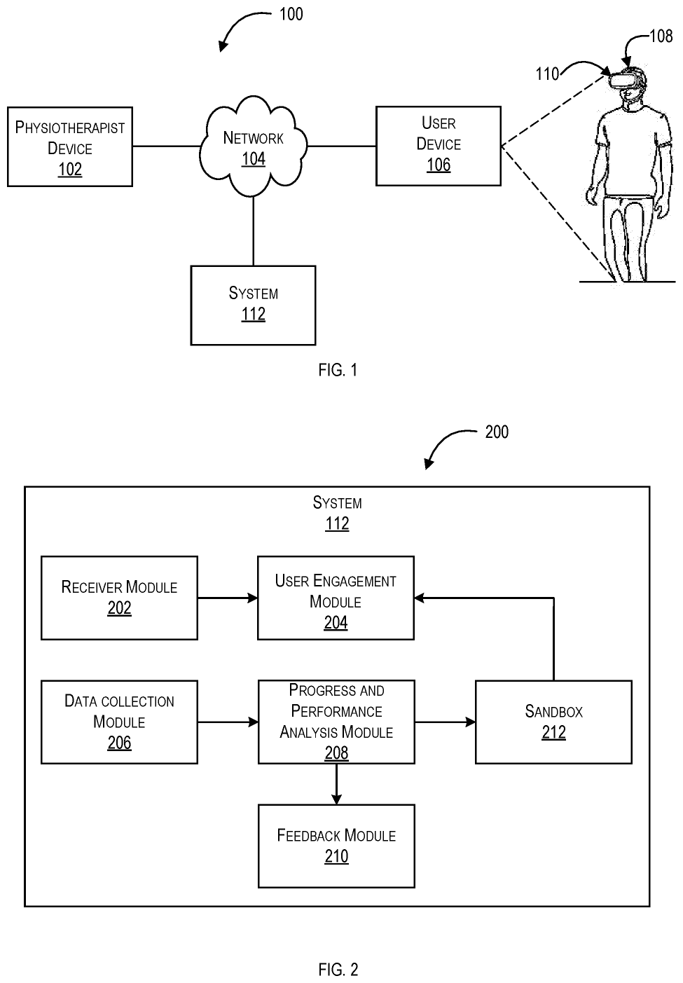

FIG. 1 illustrates a block diagram 100 of an environment of a system 112 for conducting interactive rehabilitation sessions with continuous monitoring, in accordance with various embodiments of the present disclosure. The environment may include a physiotherapist device 102, a network 104, a user device 106, a user 108, a head-mounted device 110, and the system 112 for conducting interactive rehabilitation session for the user 108. The physiotherapist device 102 and the user device 106 may correspond to an electronic device having a display screen, a camera, and speakers, such as, without any limitation, a smartphone, a smart television, a PC, a tablet, a laptop, or the like. In an embodiment, the user device 106 may be any equipment which provisions to display content rendered by the system 112 for conducting therapy session and to monitor actions of the user 108 during the session. Further, the display screen and the speakers of the user device 106 provides the content rendered by the system 112 to the user 108. In a further embodiment, the camera of the user device 106 may facilitate monitoring the actions of the user 108 during the rehabilitation session.

In an embodiment, the network 104 may include, without limitation, a direct interconnection, a Local Area Network (LAN), a Wide Area Network (WAN), a wireless network (e.g., using Wireless Application Protocol), the Internet, and the like. In an embodiment, the system 112 may be implemented as a cloud-based server that is configured to communicate with user device 106 of the user, for conducting the rehabilitation sessions. In another embodiment, the system 122 may be integral part of the user device 106 associated with the user 108. Further, the system may be configured to conduct the physiotherapy sessions for the user 106 and continuously monitor the user 108 during the sessions. When conducting the therapy sessions and monitoring the sessions, the system 112 may communicate with the user device 106 and/or the head-mounted device 110 via the network 104, such that the one or more tasks to be performed by the user 108 are rendered to the user 108 on the display screen of the device. Further, the head-mounted device 110 may correspond to a Virtual Reality (VR) device, such as a VR head-gear or smart glasses, configured to provide the user 108 a virtual environment for providing an immersive experience to the user 108 during the rehabilitation session. In scenarios where multiple users 108 are connected to the system 112 for accessing the rehabilitation session, the system 112 may be implemented as a cloud-based server communicatively connected with user devices 106 associated with the multiple users 108. In an embodiment, each of the multiple users 108 may access the rehabilitation session using dedicated user device 106 and/or the head-mounted device 110.

In an embodiment, the system 112 may compare standard parameters and specifications (such as angle, posture, movements, motions, jerks or the like) of the one or more tasks to be performed by the user 108 with the actual parameters and specifications of the user 108 in real-time. Further, if there are any deviation in the user 108 parameters and specification with the standard parameters and specification, then the system 112 may notify the user 108 for correcting such parameters and specification through one or more recommendations. Additionally, the system 112 may also provide data associated with such deviation to the physiotherapist device 102 to inform the physiotherapist about the real-time progress and mistakes of the user 108 in order to enable the physiotherapist to change or modify the one or more tasks for the user 108.

FIG. 2 illustrates a block diagram 200 of the system 112 for conducting interactive rehabilitation sessions with continuous monitoring, in accordance with various embodiments of the present disclosure.

The system 100 may include a receiver module 202, a user engagement module 204, a data collection module 206, a progress and performance analysis module 208, a feedback module 210, and a sandbox 212. The receiver module 202, the user engagement module 204, the data collection module 206, the progress and performance analysis module 208, the feedback module 210, and the sandbox 212 may be communicatively coupled to a memory and a processor of the system 112.

The processor may be configured to control the operations of the receiver module 202, the user engagement module 204, the data collection module 206, the progress and performance analysis module 208, the feedback module 210, and the sandbox 212. In an embodiment of the present disclosure, the processor and the memory may form a part of a chipset installed in the system 112. In another embodiment of the present disclosure, the memory may be implemented as a static memory or a dynamic memory. In an example, the memory may be internal to the system 112, such as an onside-based storage. In another example, the memory may be external to the system 112, such as cloud-based storage. Further, the processor may be implemented as one or more microprocessors, microcomputers, digital signal processors, central processing units, state machines, logic circuitries, and/or any devices that manipulate signals based on operational instructions.

Initially, to access the rehabilitation session, the user 108 may log into a platform of the system 112 within the user device 106. Such platform may be a software that is installed in the user device 106 to host services provided by the system 112. It may be understood that the user 108 may be an independent user without an affiliated therapist or a therapist affiliated user. In an embodiment, for independent user, health survey of the user may be conducted. For the health survey, a series of questions may be asked to the user 108 to determine user's ailments and requirements. In an embodiment, the receiver module 202 may receive one or more inputs from the user 108 in response to one or more questions associated with ailments and requirements pertaining to the user 108.

In an embodiment, the user engagement module 204 may create a customized workout plan for the user 108 with workouts for the rehabilitation session. for initiating the rehabilitation session, wherein the customized workout plan is created based on the received one or more inputs and includes one or more standard clinical features. In an embodiment, the user engagement module 204 may automatically create the customized workout plan. In another embodiment, the user engagement module 204 may report the answers to the physiotherapist via the physiotherapist device 102 and receive the physiotherapist recommendations to create the customized workout plan. In yet another embodiment, the user engagement module 204 may automatically create the customized workout plan and share the customized plan with the physiotherapist for verification.

In an embodiment, for physiotherapist affiliated users, the physiotherapist associated with the user may directly recommend the workout plans or activities for the user. The physiotherapist may initially interact with the user or may medically examine the user to provide recommendation on the workout plans. In an embodiment, the physiotherapist affiliated users may also be asked the series of questions to determine condition of the user and report with answers from the user may be provided to the physiotherapist for the recommendations. In either of the cases, the user engagement module 204 may create and render the customized workout plan to the user 108 for initiating the rehabilitation session.

In an embodiment, the customized workout plan may include one or more tasks along with specific attributes and parameters for performing the one or more tasks. The one or more tasks may be associated with sequential instructions to perform physical exercises and/or virtual objects for provisioning physiotherapy. Further, the customized workout plan may include one or more standard clinical features related to required posture and movement from the user 108 for implementing the customized workout plan. Further, the user engagement module 204 may render the created customized workout plan to the user 108. In an embodiment, the rendering may correspond to displaying the one or more tasks to the user 108 on the display screen of the user device 106. In another embodiment the rendering may correspond to creating the virtual environment for the user with one or more virtual objects through the head-mounted device 110 for performing the one or more tasks in the immersive environment.

In an embodiment, when the user 108 implements the customized workout plan by performing the one or more tasks, the data collection module 206 may receive metrics and movement data of the user 108. The metrics and movement data may, without any limitation, include speed, reaction time, angle, distance, stability, jerk, range of motion, flexibility, balance, strength, muscle power, and/or degree of flexion. Further, the data collection module 206 may be communicatively coupled to one or more sensors and/or a camera to receive the metrics and movement data of the user 108 while implementing the customized workout plan by processing the received one or more inputs. In implementation, the data collection module 206 works towards recognizing the presence of the patient/user and maps a coordinate detection system onto the user's body. The one or more sensors map this out and using this data the metrics and movement data shall be detected, and stored for further processing. In an embodiment, the data collection mechanisms may use computer vision-based body pose estimation models, to detect various exercises. It may be noted that such algorithms may be specially designed for each exercise keeping in mind the anatomical regions that are being affected.

In an embodiment, once the data has been stored and collected, the progress and performance analysis module 208 may extract one or more clinical features based on the received metrics and movement data of the user by coordinating, smoothing, normalization, dimensionality reduction, temporal correlation, windowing, feature extraction, or a combination thereof. The smoothing involves taking a sliding window of a certain size over the data and replacing each value with the average of the values within the window to reduce noise and fluctuations. The scaling of coordinate data to a common range between 0 and 1 using techniques like min-max normalization or z-score normalization ensures that the data is on a consistent scale. The dimensionality reduction includes applying techniques like principal component analysis (PCA) to reduce the dimensionality. The PCA identifies the most important components of the data and projects it onto a lower-dimensional space, if the coordinate data has a high dimensionality. The windowing divides the time series coordinate data into smaller windows or segments and compute temporal correlation measures within each window to allow capturing local temporal correlations and mean correlation is calculated between every window. The feature extraction extracts relevant features from the temporal correlation measures, such as the maximum correlation value, the number of significant peaks, and the slope of correlation changes over time. Additionally, to combine the processed features, the processed features are concatenated or merged into a single feature vector. If the coordinate data is processed, DTW scores are normalized, and temporal correlation features are extracted, they are concatenated into a single feature vector. Thus, the resulting feature vector will contain the combined information from all the processed features and this combined feature vector is then used as input for further steps such as model training or classification. The one or more clinical features may be related to the actual posture and movement of the user 108 while implementing the customized workout plan. In an embodiment, once the clinical features are extracted, the progress and performance analysis module 208 may compare the extracted one or more clinical features with the standard one or more clinical features to determine if there are deviations.

In an embodiment, the feedback module 210 may provide one or more dynamic feedbacks to the user 108 in real-time based on the determined deviations. The one or more dynamic feedbacks are provided to the user 108 by employing an explainable Artificial Intelligence (AI) model and may be associated with correction of posture and/or movement to overcome the determined deviation. The explainable AI model has been discussed in details in the following paragraphs. This will help in avoiding any injuries or wrong postures as the system 112 will alert the user 108 if they are not meeting or if they are surpassing the constraints mentioned by the physiotherapist. In implementation, the metric may be fed to a Machine Learning (ML) model which may automatically identify the wrong posture and the wrong movements user in the rehabilitation session and may indicate the user 108 on what is wrong with the posture and the movement. For example, consider the user 108 is supposed to lift the hand to 90 degrees, the progress and performance analysis module 208 may detect that is the user 108 in incorrectly performing the task (i.e., not lifting the hand to 90 degrees) and also recommend on to correct the posture or action.

In another embodiment, the progress and performance analysis module 208 may create a report based on the results of the comparison and sends the report to the user 108 and the physiotherapist of the user 108. Additionally, based upon these reports and metrics, the physiotherapist may modify the treatment plan or any other exercise constraints. In an embodiment, the sandbox 212 may facilitate the physiotherapist to create a new custom exercise with customized constraints for modifying the workout plan of the user 108 by uploading a video performing the new custom exercise and adding body point of interest, relevant angles, and/or necessary metrics for tracking recovery progress of the user 108 in the rehabilitation session. In implementation, once the modified workout plan set by the physiotherapist is sent to the user 108, the modified workout plan shall appear in the user's dashboard. In an embodiment, a demo of the one or more associated tasks may be displayed to the user 108 so that the user 108 can follow the one or more tasks and perform accordingly. In an embodiment, the sandbox 212 may employ a freeze frame method to facilitate the physiotherapist to first upload the video and then pick particular frames to assign the name of the movement being performed to the frame. As a result, the physiotherapist may confirm that the detected angles and range are accurate while some labels such as starting position and ending position may be specified to help generate the new exercise. In implementation, the physiotherapist may drag and drop other exercises to be added in between and also specify the number of repetitions for each exercise.

In an embodiment, the user engagement module 204 may be communicatively coupled to a visualization module (not shown) that may built scenarios and real-life virtualization of the one or more tasks for the rehabilitation session. The visualization module may create real life scenes/scenarios and objects, and characters may be replicated in the one or more tasks. In an embodiment, the created scenarios may, without any limitation, depict daily life tasks such as bursting balloons in a playground, picking up flowers from a garden, playing handball, or the like. In an embodiment, the visualization module may facilitate the physiotherapist to create their own set of virtual objects that they would want the patient to interact with and may customize the size, color, speed of the virtual objects while providing the game metrics such as overall score, reaction time, accuracy of identifying objects.

In an embodiment, the progress and performance analysis module 208 may be communicatively coupled to a facial emotion module to recognize the facial emotions of the user 108 while performing the one or more task, and focusing on interacting and recognizing the voice of the user 108. Since, facial emotion recognition may allow calculation of a level of pain being faced by the user 108 via monitoring facial features. Such data may also be recorded and shared in the report with the physiotherapist. Additionally, such data may also be used to monitor when there are changes in emotions particularly in neurological therapy which would help doctors better identify any triggers.

In an embodiment, the feedback module 210 may be communicatively coupled to a voice recognition module to inform the user 108 performing the exercise of any bad postures, incorrect movements and to help inform them of their progress. Additionally, the user 108 may be facilitated with a provision to speak to a voice assistant and ask questions regarding the workout/progress and receive answers through the speakers of the user device 106. This would be particularly useful for geriatric care, which involved working with the elderly, who may feel more comfortable with voice assistants and may feel motivated due to the same. This also helps in scenarios where the user 108 is performing exercises while being far or turned away from the device screen. Further, the system 112 may be configured to function in multiple languages that can increase reachability with the users.

In an additional embodiment, the system 112 may include a color code representation module to use color code representation to assist the user in understanding whether they are performing the exercise accurately. In an embodiment, the colors such as red, blue and green may be used. Red may highlight particular join/limb of the user that is performing incorrectly and should correct it to proceed further. Green may be used to highlight that the user is performing the exercise accurately and can proceed further. Blue may be used to depict that the user is in mid-way of performing a particular action and may proceed further in order to complete the action.

In an embodiment, the explainable AI may consider different classes of incorrect body movements, which may be categorized into different sub-classes depending upon the type and severity of the incorrect movement. Further, data points related to the body movements of an individual may be provided as inputs to the model to predict the class of the incorrect movement. Further, the classification model may work by first analyzing the input data and extracting relevant features that may be essential for identifying the incorrect movement. Such features would then be fed into the Graph Convolutional Networks or Neural Network model, that may be trained to classify the input data into the different classes of incorrect movements. In an embodiment, the accuracy of the model may be be determined by how well it can distinguish between different types of incorrect movements and correctly categorize them into the corresponding classes. Incorporating explainable AI into this classification model may be crucial as it may help in providing an understanding of how the model arrived at a particular classification decision. It may be noted that the explainable AI may explain the decision-making process in a human-understandable language and may be particularly important when dealing with medical data or any other sensitive data, as it helps in ensuring transparency and accountability. In order to incorporate explainable AI into the classification model, one or more different techniques may be used such as LIME (Local Interpretable Model-Agnostic Explanations) or SHAP (SHapley Additive exPlanations) or CAM (Class activation Mapping). These techniques help in understanding the importance of different features and how they contribute to the final classification decision. The CAM may help identify and highlight exact points in the image that contributed to a particular incorrect classification. This information may be presented to the end-users in the form of graphs or charts, which may help them understand the reasoning behind the model's decision.

In an embodiment, the classification model may receive three different types of inputs/parameters based upon which it makes the classification. The model may receive coordinate data of 33 different biomarkers of the body, the dynamic time warping score of the current data compared to the ideal data, as well as the temporal correlation using time series analysis of the current data (exercise) with respect to an ideally performed exercise. These biomarkers capture the position and movement of joints, limbs, or other relevant body parts involved in the exercise. The coordinate data provides information about the spatial aspects of the movement. The model also receives the DTW score, which quantifies the similarity or dissimilarity between the current movement and an ideal or reference movement. The DTW algorithm aligns and compares the sequences of feature vectors extracted from the time series coordinate data. The resulting score reflects the alignment or matching between the current movement and the ideal movement. A lower DTW score indicates a closer match to the ideal movement. Additionally, the model takes into account the temporal correlation of the current movement with respect to an ideally performed exercise. This involves analyzing the time series coordinate data to identify patterns, dynamics, or deviations from the ideal movement pattern. The temporal correlation provides insights into the synchronization and timing aspects of the movement. This model can be considered as a supervised learning approach, where the training data has been divided into different “incorrect exercise/movement” categories. This dataset has been collected over time with various numbers of incorrect movement categories and varied body types. The labeled training data includes examples of correct movements as well. By contrasting the incorrect movements with the correct ones, the model learns to differentiate between them. During the training process, the model optimizes its internal parameters based on the input data and corresponding labels. The model learns to extract meaningful features from the coordinate data, DTW scores, and temporal correlation. These features capture important aspects of the movement that contribute to its correctness or incorrectness. The model then uses these features to classify the movements into different categories of incorrectness.

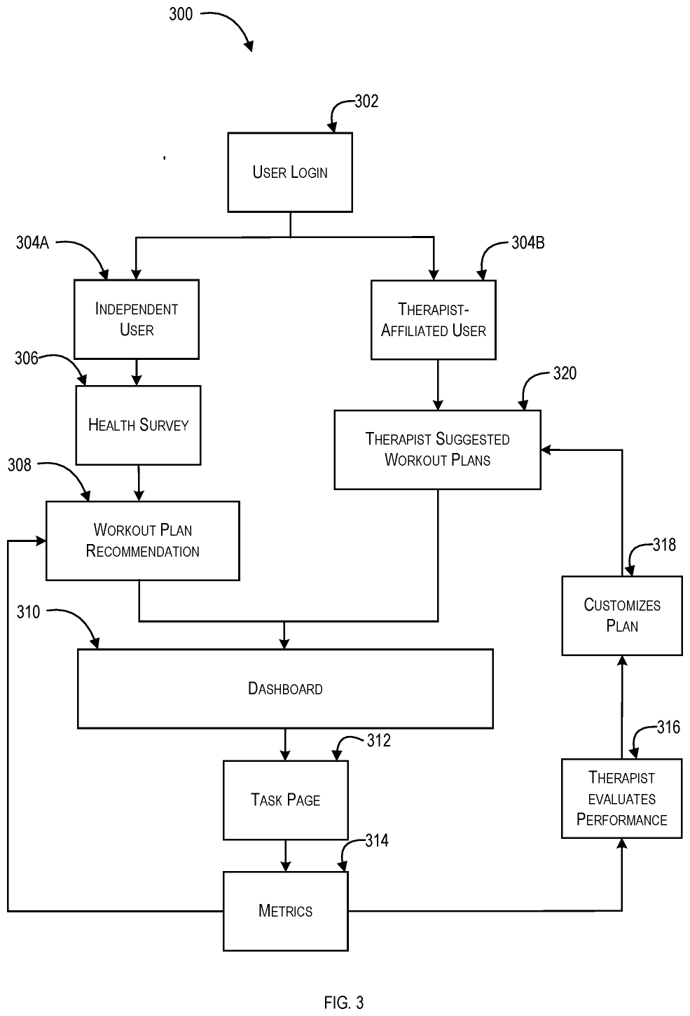

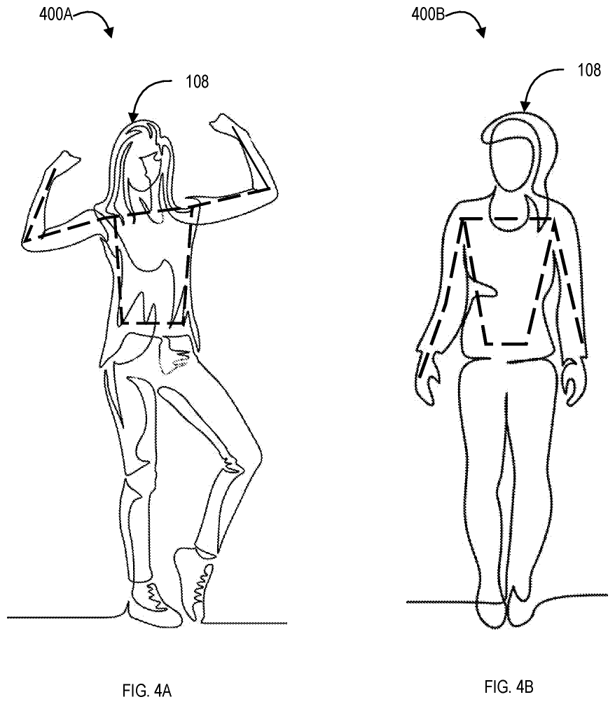

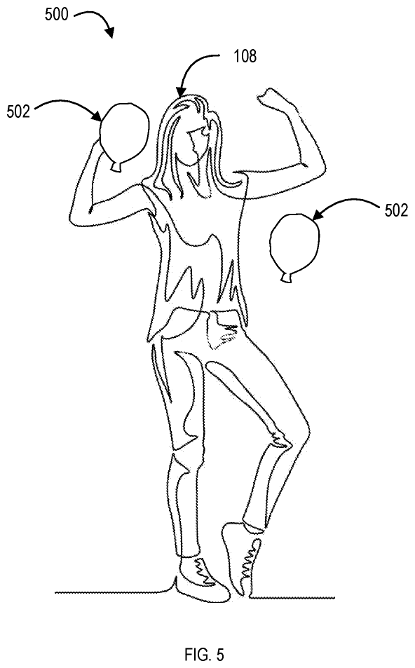

FIG. 3 illustrates an overall working mechanism 300 for the system 112 for conducting interactive rehabilitation sessions with continuous monitoring, in accordance with various embodiments of the present disclosure. FIGS. 4A-4B illustrate an exemplary embodiment of a task for conducting interactive physiotherapy session with continuous monitoring, in accordance with various embodiments of the present disclosure. FIG. 5 illustrate another exemplary embodiment of a task for conducting interactive physiotherapy session with continuous monitoring, in accordance with various embodiments of the present disclosure. For the sake of brevity, FIGS. 3, 4A-4B, and 5 have been explained together.

Initially, to access the rehabilitation session, the user 108 may log into platform of the system 112 within the user device 106, as shown by box 302. If the user 108 is an independent user, as shown by 304A, then the system 112, may conduct a health survey of the user 108 to ask a series of questions for determining patient's ailments and requirements. Based on the inputs from the user 108 in response to the series of questions, the system 112 may recommend a workout plan recommendation to the user 108, as shown by box 308. Such workout plans may be created automatically by the system 112. If the user 108 is a therapist-affiliated user, as shown by box 304B, then the system 112 may alternatively, or additionally request the physiotherapist to recommend a plan to the user 108. Therefore, in such a scenario, the workout plan for the user 108 may be created automatically by the system 112, by the physiotherapist, or created by the system 112 and verified by the physiotherapist.

Upon creation of the workout plan for the user 108, the created workout plan may be sent to the user device 106. When the recommended workout plan is received by the user device 106, such workout plan may be displayed on dashboard of the user device 106, as shown by 310. Upon selection of the displayed workout plan, the user 108 may be taken to a task page, as shown by 312, where one or more tasks associated with the workout plan may be displayed to the user 108 on the user device 106. In another embodiment, the workout plan may be displayed in forms of the one or more tasks to the user 108. In yet another embodiment, the system 112 may store the one or more tasks for multiple workout plans which may be recommended to the user 108. Therefore, when a workout plan is recommended to the user, the one or more tasks may be selected for the user to conduct the rehabilitation session.

In an embodiment, as shown in FIGS. 4A and 4B, a task from the one or more tasks may include sequential instructions provided to the user 108 to perform physical exercises like to lift arms up and down, as shown by 400A and 400B, respectively. In another embodiment, as shown in FIG. 5, when the user 108 opts for implementing the workout plan, then the system 112 may create a virtual environment with one or more virtual objects 502 for the user 108 via the head-mounted 110, such that the user 108 may perform the one or more tasks in an immersive environment. For example, a task of the one or more tasks may include performing actions along with a virtual balloon or busting the virtual balloons.

In an embodiment, the task may commence immediately or may be scheduled for a later date or time. Once the user 108 clicks on the task, the user 108 may be guided to a task page which starts monitoring actions of the user 108. During the implementation of the workout plan or performing of the one or more tasks, the system 112 may monitor and capture one or more real-time metrics (such as angle, distance of the actions, or speed of the actions performed of the user 108) associated with the user 108, as shown by box 314. In an embodiment, the one or more metrics may be used to determine if the user 108 is performing the one or more tasks correctly or not based upon one or more criteria which are preset by the physiotherapists. In case, the user 108 is not performing the one or more tasks correctly, then the system 112 may give feedbacks to the user for correcting the associated pose, motion, and/or movement to correctly perform the one or more tasks. Once the one or more tasks are completed, the user may receive a report mentioning their progress, tasks well done, tasks missed and other analytics.

In an embodiment, the report may also be shared with the physiotherapist and the physiotherapist may evaluate the performance of the user 108, as shown in box 316. The evaluation may, without any limitation, be based on goals achieved and the user's progress is provided in the report. Further, the report may also include a few screenshots of the user 108 performing the actions in accordance with the one or more tasks as well as a document with any error logs. Based upon the evaluation, the physiotherapist may alter the series of tasks that have been assigned and any other customizations that may be required to customize the workout plan, as shown in box 318. The customizations may be associated with changing speed, duration, number of repetitions, angles that need to be achieved between joints of the user 108 during the session, and so on.

In an embodiment, an alert system may be designed to notify the first point of contact/emergency contact when there is no movement shown by the patient and/or when a fall is detected by the camera while performing the exercise. The alert system has a gesture recognition system such that when the patient shows the gesture to the camera the emergency contact will be notified immediately. Further, if the patient consistently performs incorrectly, the exercise will be paused, patient will be notified, and after a countdown the exercise will resume once more. This prevents patient from over exerting themselves or hurting themselves by performing incorrectly.

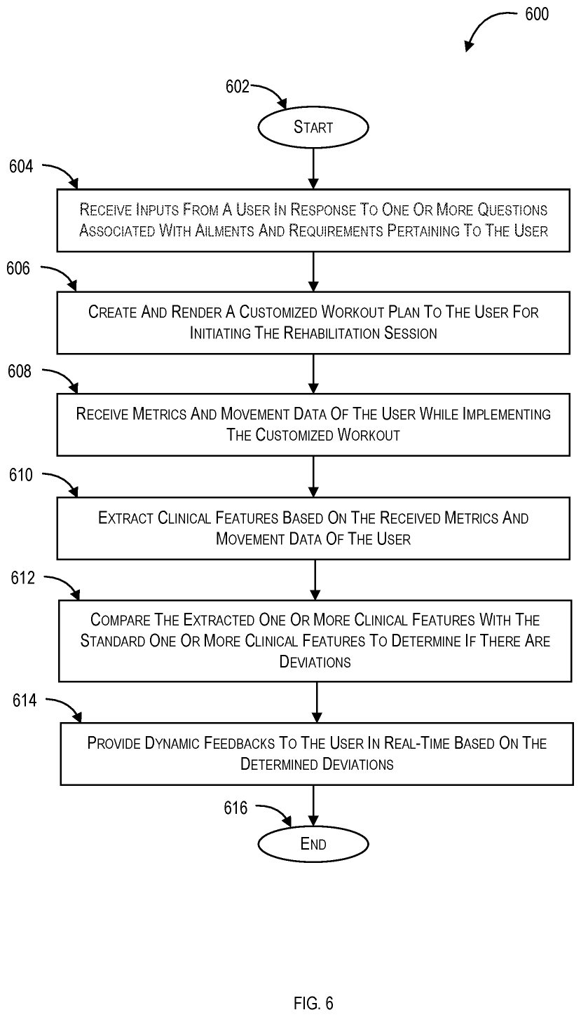

FIG. 6 illustrates a flowchart 600 of a method for conducting interactive rehabilitation sessions with continuous monitoring, in accordance with an embodiment of the present disclosure. The method starts at step 602.

At first, at step 604, one or more inputs from a user may be received. The one or more inputs may be received receiving via a user device in response to one or more questions associated with ailments and requirements pertaining to the user. Further, at step 606, a customized workout plan may be created and rendered to the user for initiating the rehabilitation session. The customized workout plan may be automatically created or created by a physiotherapist based on the received one or more inputs and includes one or more standard clinical features related to required posture and movement from the user for implementing the customized workout plan. Further, the customized workout plan may include one or more tasks along with specific attributes and parameters for performing the one or more tasks associated with sequential instructions to perform physical exercises and/or virtual objects for provisioning physiotherapy. In an embodiment, the customized workout plan may be rendered in a virtual environment and may include one or more virtual objects to facilitate interaction with the user to make the rehabilitation session immersive.

Further, at step 608, metrics and movement data of the user while implementing the customized workout plan during the rehabilitation session may be received via one or more sensors and/or a camera while implementing the customized workout plan by processing the received one or more inputs. Further, the metrics and movement data may, without any limitation, include speed, reaction time, angle, distance, stability, jerk, range of motion, flexibility, balance, strength, muscle power, and degree of flexion. After that, at step 610, one or more clinical features may be extracted based on the received metrics and movement data of the user by coordinating, smoothing, normalization, dimensionality reduction, temporal correlation, windowing, feature extraction, or a combination thereof. The one or more clinical features may be related to actual posture and movement of the user while implementing the customized workout plan.

Next, at step 612, the extracted one or more clinical features may be compared with the standard one or more clinical features to determine if there are deviations. Thereafter, at step 614, one or more dynamic feedbacks may be provided to the user in real-time based on the determined deviations by employing an explainable Artificial Intelligence (AI) model. The one or more dynamic feedbacks are associated with correction of posture and/or movement to overcome the determined deviation.

In an embodiment, the method also includes the steps of creating a report based on the results of the comparison and sending the report to at least one of: the user and a physiotherapist of the user. Further, the method includes the steps of facilitating the physiotherapist to create a new custom exercise with customized constraints to modify the workout plan of the user by uploading a video performing the new custom exercise and adding body point of interest, relevant angles, and/or necessary metrics for tracking recovery progress of the user in the rehabilitation session. The method ends at step 616.

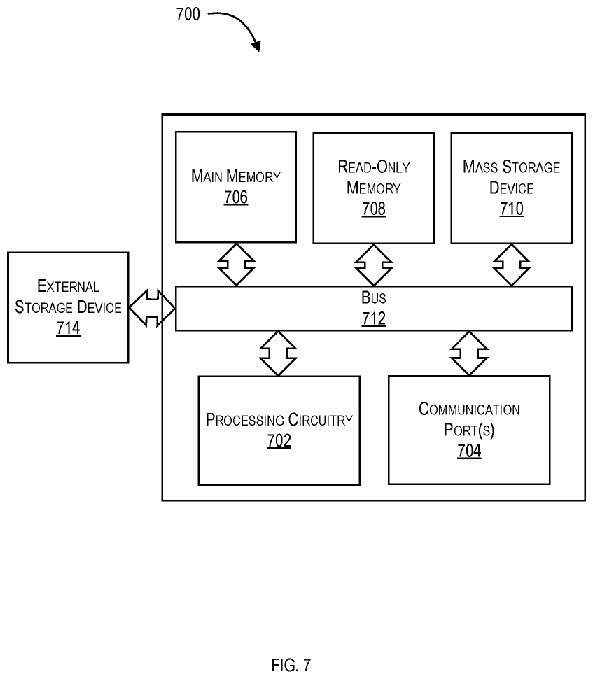

FIG. 7 illustrates an exemplary computer system in which or with which embodiment of the present disclosure may be utilized. As shown in FIG. 7, a computer system 700 includes an external storage device 714, a bus 712, a main memory 706, a read-only memory 708, a mass storage device 710, a communication port 704, and a processor 702.

Those skilled in the art will appreciate that computer system 700 may include more than one processor 702 and communication ports 704. Examples of processor 702 include, but are not limited to, an Intel® Itanium® or Itanium 2 processor(s), or AMD® Opteron® or Athlon MP® processor(s), Motorola® lines of processors, FortiSOC™ system on chip processors or other future processors. Processor 702 may include various modules associated with embodiments of the present disclosure.

Communication port 704 can be any of an RS-232 port for use with a modem-based dialup connection, a 10/100 Ethernet port, a Gigabit or 10 Gigabit port using copper or fiber, a serial port, a parallel port, or other existing or future ports. Communication port 704 may be chosen depending on a network, such as a Local Area Network (LAN), Wide Area Network (WAN), or any network to which the computer system 700 connects.

Memory 706 can be Random Access Memory (RAM), or any other dynamic storage device commonly known in the art. Read-Only Memory 708 can be any static storage device(s) e.g., but not limited to, a Programmable Read-Only Memory (PROM) chips for storing static information e.g., start-up or BIOS instructions for processor 702.

Mass storage 710 may be any current or future mass storage solution, which can be used to store information and/or instructions. Exemplary mass storage solutions include, but are not limited to, Parallel Advanced Technology Attachment (PATA) or Serial Advanced Technology Attachment (SATA) hard disk drives or solid-state drives (internal or external, e.g., having Universal Serial Bus (USB) and/or Firewire interfaces), e.g. those available from Seagate (e.g., the Seagate Barracuda 7200 family) or Hitachi (e.g., the Hitachi Deskstar 7K1000), one or more optical discs, Redundant Array of Independent Disks (RAID) storage, e.g. an array of disks (e.g., SATA arrays), available from various vendors including Dot Hill Systems Corp., LaCie, Nexsan Technologies, Inc. and Enhance Technology, Inc.

Bus 712 communicatively couples processor(s) 702 with the other memory, storage, and communication blocks. Bus 712 can be, e.g., a Peripheral Component Interconnect (PCI)/PCI Extended (PCI-X) bus, Small Computer System Interface (SCSI), USB, or the like, for connecting expansion cards, drives, and other subsystems as well as other buses, such a front side bus (FSB), which connects processor 702 to a software system.

Optionally, operator and administrative interfaces, e.g., a display, keyboard, and a cursor control device, may also be coupled to bus 712 to support direct operator interaction with the computer system. Other operator and administrative interfaces can be provided through network connections connected through communication port 704. An external storage device 714 can be any kind of external hard-drives, floppy drives, IOMEGA® Zip Drives, Compact Disc-Read-Only Memory (CD-ROM), Compact Disc-Re-Writable (CD-RW), Digital Video Disk-Read Only Memory (DVD-ROM). The components described above are meant only to exemplify various possibilities. In no way should the aforementioned exemplary computer system limit the scope of the present disclosure.

While embodiments of the present disclosure have been illustrated and described, it will be clear that the disclosure is not limited to these embodiments only. Numerous modifications, changes, variations, substitutions, and equivalents will be apparent to those skilled in the art, without departing from the spirit and scope of the disclosure, as described in the claims.

Thus, it will be appreciated by those of ordinary skill in the art that the diagrams, schematics, illustrations, and the like represent conceptual views or processes illustrating systems and methods embodying this disclosure. The functions of the various elements shown in the figures may be provided through the use of dedicated hardware as well as hardware capable of executing associated software. Similarly, any switches shown in the figures are conceptual only. Their function may be carried out through the operation of program logic, through dedicated logic, through the interaction of program control and dedicated logic, or even manually, the particular technique being selectable by the entity implementing this disclosure. Those of ordinary skill in the art further understand that the exemplary hardware, software, processes, methods, and/or operating systems described herein are for illustrative purposes and, thus, are not intended to be limited to any particular named.

As used herein, and unless the context dictates otherwise, the term “coupled to” is intended to include both direct coupling (in which two elements that are coupled to each other contact each other) and indirect coupling (in which at least one additional element is located between the two elements). Therefore, the terms “coupled to” and “coupled with” are used synonymously. Within the context of this document terms “coupled to” and “coupled with” are also used euphemistically to mean “communicatively coupled with” over a network, where two or more devices can exchange data with each other over the network, possibly via one or more intermediary device.

It should be apparent to those skilled in the art that many more modifications besides those already described are possible without departing from the inventive concepts herein. The inventive subject matter, therefore, is not to be restricted except in the spirit of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. Where the specification claims refer to at least one of something selected from the group consisting of A, B, C . . . and N, the text should be interpreted as requiring only one element from the group, not A plus N, or B plus N, etc.

While the foregoing describes various embodiments of the disclosure, other and further embodiments of the disclosure may be devised without departing from the basic scope thereof. The scope of the disclosure is determined by the claims that follow. The disclosure is not limited to the described embodiments, versions, or examples, which are included to enable a person having ordinary skill in the art to make and use the disclosure when combined with information and knowledge available to the person having ordinary skill in the art.

We claim:

1. A system for conducting interactive rehabilitation sessions with continuous monitoring, the system comprising:
a receiver module to receive, via a user device, one or more inputs from a user in response to one or more questions associated with ailments and requirements pertaining to the user;
a user engagement module to create and render a customized workout plan to the user for initiating the rehabilitation session, wherein the customized workout plan is created based on the received one or more inputs and includes one or more standard clinical features;
a data collection module to receive metrics and movement data of the user while implementing the customized workout plan during the rehabilitation session;
a progress and performance analysis module to:
extract one or more clinical features based on the received metrics and movement data of the user by at least one of: coordinating, smoothing, normalization, dimensionality reduction, temporal correlation, windowing, and feature extraction; and
compare the extracted one or more clinical features with the standard one or more clinical features to determine if there are deviations; and
a feedback module to provide, by employing an explainable Artificial Intelligence (AI) model, one or more dynamic feedbacks to the user in real-time based on the determined deviations, wherein the one or more dynamic feedbacks are associated with correction of at least one of: posture and movement to overcome the determined deviation.

2. The system of claim 1, wherein the customized workout plan is at least one of: automatically created and created by a physiotherapist.

3. The system of claim 1, wherein the customized workout plan includes one or more tasks along with specific attributes and parameters for performing the one or more tasks.

4. The system of claim 3, wherein the one or more tasks are associated with at least one of: sequential instructions to perform physical exercises and virtual objects for provisioning physiotherapy.

5. The system of claim 1, wherein the data collection module is communicatively coupled to at least one of: one or more sensors and a camera to receive the metrics and movement data of the user while implementing the customized workout plan by processing the received one or more inputs.

6. The system of claim 1, wherein the metrics and movement data include at least one of: speed, reaction time, angle, distance, stability, jerk, range of motion, flexibility, balance, strength, muscle power, and degree of flexion.

7. The system of claim 1, wherein the one or more standard clinical features are related to required posture and movement from the user for implementing the customized workout plan, and the one or more clinical features are related to actual posture and movement of the user while implementing the customized workout plan.

8. The system of claim 1, wherein the customized workout plan is rendered in a virtual environment and includes one or more virtual objects to facilitate interaction with the user to make the rehabilitation session immersive.

9. The system of claim 1, wherein the progress and performance analysis module creates a report based on the results of the comparison and sends the report to at least one of: the user and a physiotherapist of the user.

10. The system of claim 9, further comprises a sandbox to facilitate the physiotherapist to create a new custom exercise with customized constraints to modify the workout plan of the user by uploading a video performing the new custom exercise and adding at least one of: body point of interest, relevant angles, and necessary metrics for tracking recovery progress of the user in the rehabilitation session.

11. A method for conducting interactive rehabilitation sessions with continuous monitoring, the method comprising:
receiving, via a user device, one or more inputs from a user in response to one or more questions associated with ailments and requirements pertaining to the user;
creating and rendering a customized workout plan to the user for initiating the rehabilitation session, wherein the customized workout plan is created based on the received one or more inputs and includes one or more standard clinical features;
receiving metrics and movement data of the user while implementing the customized workout plan during the rehabilitation session;
extracting one or more clinical features based on the received metrics and movement data of the user by at least one of: coordinating, smoothing, normalization, dimensionality reduction, temporal correlation, windowing, and feature extraction;
comparing the extracted one or more clinical features with the standard one or more clinical features to determine if there are deviations; and
providing, by employing an explainable Artificial Intelligence (AI) model, one or more dynamic feedbacks to the user in real-time based on the determined deviations, wherein the one or more dynamic feedbacks are associated with correction of at least one of: posture and movement to overcome the determined deviation.

12. The method of claim 11, wherein the customized workout plan is at least one of: automatically created and created by a physiotherapist.

13. The method of claim 11, wherein the customized workout plan includes one or more tasks along with specific attributes and parameters for performing the one or more tasks.

14. The method of claim 13, wherein the one or more tasks are associated with at least one of: sequential instructions to perform physical exercises and virtual objects for provisioning physiotherapy.

15. The method of claim 11, wherein the metrics and movement data of the user is received via at least one of: one or more sensors and a camera while implementing the customized workout plan by processing the received one or more inputs.

16. The method of claim 11, wherein the metrics and movement data include at least one of: speed, reaction time, angle, distance, stability, jerk, range of motion, flexibility, balance, strength, muscle power, and degree of flexion.

17. The method of claim 11, wherein the one or more standard clinical features are related to required posture and movement from the user for implementing the customized workout plan, and the one or more clinical features are related to actual posture and movement of the user while implementing the customized workout plan.

18. The method of claim 11, wherein the customized workout plan is rendered in a virtual environment and includes one or more virtual objects to facilitate interaction with the user to make the rehabilitation session immersive.

19. The method of claim 11, further comprises:
creating a report based on the results of the comparison; and
sending the report to at least one of: the user and a physiotherapist of the user.

20. The method of claim 19, further comprises facilitating the physiotherapist to create a new custom exercise with customized constraints to modify the workout plan of the user by uploading a video performing the new custom exercise and adding at least one of: body point of interest, relevant angles, and necessary metrics for tracking recovery progress of the user in the rehabilitation session.
